(19) United States
(12) Patent Application Publication
(10) Pub. No.: US 2010/0305004 A1
(43) Pub. Date: Dec. 2, 2010

(54) POLYNUCLEOTIDE BACKBONES FOR COMPLEXING PROTEINS

(75) Inventors: Michael P. Weiner, Guilford, CT (US); Michael I. Sherman, Glen Ridge, NJ (US)

Correspondence Address: BANNER & WITCOFF, LTD., 1100 13th STREET, N.W., SUITE 1200, WASHINGTON, DC, 20005-4051, US

(73) Assignee: AFFOMIX CORPORATION, Branford, CT (US)

(21) Appl. No.: 12/679,586

(22) PCT Filed: Sep. 26, 2008
(86) PCT No.: PCT/US08/77887

§ 371 Date: Aug. 13, 2010

Related U.S. Application Data
(60) Provisional application No. 60/975,974, filed on Sep. 28, 2007.

(51) Int. Cl. C40B 50/06; C07K 14/00; C12N 9/00; G01N 33/50

(52) U.S. Cl. 506/26; 530/358; 435/183; 435/7.1

(57) ABSTRACT

We use the Tus-Ter interaction to enable the utilization of nucleic acid analytical methodologies for proteins. We also use the Tus-Ter interaction to make polymers and oligomers that have a nucleic acid backbone with protein functionalities. These methods are useful for molecular modeling, for efficiently running enzymatic pathway reactions, and for analyzing presence and/or amount of particular proteins.

### TECHNICAL FIELD OF THE INVENTION

This invention is related to the area of protein-nucleic acid complexes. In particular, it relates to making and using such complexes for analytic, synthetic, and therapeutic purposes.

### BACKGROUND OF THE INVENTION

Essential to the ambition of fully characterizing the human proteome are systematic and comprehensive collections of specific affinity reagents directed against all human proteins, including splice variants and modifications. Although a large number of affinity reagents are available commercially, their quality is often questionable and only a fraction of the proteome is covered. In order for more targets to be examined, there is a need for broad availability of panels of affinity reagents, including binders to proteins of unknown functions. In addition to the formidable task of assembling these reagents are the challenges of developing an inexpensive and facile means for using them.

There is a continuing need in the art to create affinity reagents for interrogating the proteome. There is a continuing need in the art for manipulable backbone structures for combining proteins and protein domains. There is a continuing need in the art for arrays for interrogating the proteome. There is a continuing need in the art for methods for quantitating proteins over a wide range of concentrations. There is a continuing need in the art for protein immobilization techniques in which the proteins retain biological activity. These and other needs are met as described below.

### SUMMARY OF THE INVENTION

According to one embodiment, a polymer is provided. The polymer comprises a plurality of monomers. Each monomer comprises a non-covalent complex of a fusion protein and a nucleic acid molecule. The fusion protein comprises a Tus protein and a polypeptide and the nucleic acid molecule comprises a Ter site.

According to another embodiment a method of assembling a polymer is provided. A plurality of monomers are ligated to each other using a DNA ligase enzyme. Each monomer comprises a non-covalent complex of a fusion protein, and a nucleic acid molecule. The fusion protein comprises a Tus protein and a polypeptide, and the nucleic acid molecule comprises a Ter site.

In yet another embodiment, a protein-DNA complex is provided. The complex comprises a fusion protein and a nucleic acid molecule. The fusion protein comprises a Tus protein and a binding polypeptide. A first portion of the nucleic acid molecule is double stranded and a second portion of the nucleic acid molecule is single stranded. The first portion comprises a Ter sequence and the second portion comprises an addressing sequence. Each addressing sequence is complexed with a fusion protein comprising a unique binding polypeptide.

Also provided is an arrayed library of binding polypeptides. Each binding polypeptide is tethered to a substratum using non-covalent binding of a Tus protein to a Ter sequence. Each binding polypeptide is fused to a Tus protein. Each Ter sequence is in a nucleic acid molecule comprising double and single stranded portions. The single stranded portions comprise an addressing sequence and the double stranded portions comprise the Ter sequence. The addressing sequence is complementary to a single stranded probe which is attached to the substratum.

Another aspect is a method to measure a target molecule. The target molecule is bound by two distinct binding polypeptides. A first and a second binding polypeptide are mixed with a target molecule to form a mixture. Each binding polypeptide is part of a fusion protein with a Tus protein and the Tus protein is bound to a DNA molecule which comprises a double-stranded portion and a single-stranded portion. The double-stranded portion comprises a Ter sequence, and the single stranded portion comprises a tag sequence which uniquely corresponds to the binding polypeptide. A bridging oligonucleotide is added to the mixture under conditions in which complementary DNA single strands will form double strands. The bridging oligonucleotide comprises a first and a second portion. The first portion is complementary to the tag sequence of the first binding polypeptide and the second portion is complementary to the tag sequence of the second binding polypeptide. The first and the second portions of the bridging oligonucleotide are separated by 0 to 6 nucleotides. DNA ligase is added to the mixture; the ligase joins 5′ and 3′ ends of nicked double-stranded DNA molecules. Ligated molecules comprising the first and second tag sequences and the ligation junction are amplified, forming an amplified analyte DNA strand. An assay is performed to determine amount in the mixture of the analyte DNA strand. The amount of the analyte DNA molecule is related to the amount of the target molecule.

A method for attaching an enzyme to a substratum is also provided. A nucleic acid molecule is attached to a substratum by means of covalent or non-covalent coupling. The nucleic acid molecule comprises a Ter sequence. The nucleic acid molecule previously formed or subsequently forms a complex with a fusion protein that comprises a Tus protein and an enzyme.

A method is also provided for forming an arrayed library of diverse protein-DNA complexes. One or more substrata comprising single stranded probes and a library of diverse protein-DNA complexes are mixed together. Each complex comprises a fusion protein and a nucleic acid molecule. The fusion protein comprises a Tus protein and a binding polypeptide. A first portion of the nucleic acid molecule is double stranded and a second portion of the nucleic acid molecule is single stranded. The first portion comprises a Ter sequence and the second portion comprises an addressing sequence. Each addressing sequence is complexed with a fusion protein comprising a unique binding polypeptide. The single stranded probes each comprise a sequence of at least 6 nucleotides which is complementary to an addressing sequence in the nucleic acid molecules. Upon mixing, the protein-DNA complexes bind to single stranded probes having complementary sequences by hybridization.

A method of assembling a polymer is provided. A first and a second fusion protein are mixed with a nucleic acid molecule which is pre-bound to a third fusion protein. The nucleic acid molecule comprises at least three Ter sites. Each fusion protein comprises a Tus protein and a polypeptide. The first and second fusion proteins comprise a first and second scFv fragment as the polypeptide. The third fusion protein comprises an Fc fragment of an immunoglobulin molecule as the polypeptide.

Finally, another method of assembling a polymer is provided. The polymer comprises a plurality of fusion proteins. A nucleic acid molecule is mixed with a plurality of fusion proteins. Each fusion protein comprises a Tus protein and a polypeptide. The nucleic acid molecule comprises a sufficient number of Ter sites to bind a desired number of fusion proteins.

These and other embodiments which will be apparent to those of skill in the art upon reading the specification provide the art with tools and reagents for manipulating protein molecules with the sophisticated analytic and synthetic techniques of nucleic acids.

### BRIEF DESCRIPTION OF THE DRAWINGS

FIG. 1A-1B. Flow chart of disclosure. (FIG. 1A) DNA-directed immobilization can be used to create self-assembling protein chips. A fusion of the Tus protein with either green fluorescent protein (GFP) or an scFv monoclonal antibody (as shown) can be incubated with an oligonucleotide comprising a Ter sequence and an additional approximately 21 nt-long single-stranded DNA “ZipCode” to create a Tus-fusion:TerB-ZipCode complex. After removal of unincorporated TerB, this complex can be bound to a complementary ZipCode (cZipCode) fixed to a solid surface [either an Affymetrix chip™ (as shown) or Luminex™-type bead]. DNA ligase may be used to covalently bind the complex to the array substratum. (FIG. 1B) The proximity ligation assay (PLA) reaction and formation of proximity probes. Paired proximity probes (in this case antibodies each fused to Tus) that bind to different epitopes of the same antigen can be combined with sample in a reaction tube. Upon binding to the same cognate antigen, the two proximity probes are brought close together so that the proximity probes (identified as 1 and 2 in the figure) can hybridize to a bridge oligonucleotide. Reagents necessary for the ligation and PCR step are added, and proximity probes 1 and 2 are ligated together, forming a new sequence (P1-ZipCode1-ZipCode2-P2) that can be amplified and detected by either real-time PCR or in multiplex format by hybridization to sequences complementary to the ZipCodes on a DNA microarray.

FIG. 2. Antibody Structure. (Left) The simplest antibody (IgG) comprises four polypeptide chains, two heavy (H) chains and two light (L) chains containing variable (V-regions) inter-connected by disulphide bonds [Huston, 2001]. Each V region is made up from three CDRs separated by four framework regions. The CDRs are the most variable part of the variable regions, and they perform the critical antigen binding function. The CDR regions are derived from many potential germ line sequences via a complex process involving recombination, mutation and selection. (Right) The function of binding antigens can be performed by fragments of a whole antibody. An example of a binding fragment is the Fv fragment consisting of the VL and VH domains of a single arm of an antibody. (Bottom) Although the two domains of the Fv fragment are coded for by separate genes, it has been proven possible to make a synthetic linker that enables the domains to be made as a single protein chain (known as a single chain Fv (scFv); [Bird, 1988; Huston, 1988] by recombinant methods.

FIG. 3A-3B. Standard monoclonal Ab production by mouse hybridoma and phage display. (FIG. 3A) Production of Monoclonal Antibodies by Hybridoma Technology. Immunization of animals with a selected antigen stimulates antibody-forming immune cells to produce a range of antibodies with varying specificities and potencies. Collections of immune cells are fused with tumor (myeloma) cells to produce immortalized hybridoma cells, each with a distinctive reactivity. These hybridoma cells are then screened in vitro for those with reactivities against the antigen of interest, and specific clones are isolated by limiting dilution. These cells are grown by clonal expansion, and a single population of mAb is harvested. (FIG. 3B) M13 Bacteriophage Biopanning. Sequential panning and infection cycles are carried out to enrich for phage that bind to the “bait” attached to the solid support. The phagemids are rescued in E. coli and individual picks can be assayed by superinfection with M13 helper phage to produce phage for a 96-well ELISA (enzyme-linked immunosorbent assay).

FIG. 4. Structure of the Tus:Ter complex (Kamada 1996). The position of the four mutated residues and the orientation of the permissive and nonpermissive faces of the complex are shown. The four α-strands of the central DNA-binding domain wind around the back of the DNA helix, in the major groove, between the two domains. The rings indicate the strands which pass through the central channel of the approaching DnaB helicase.

FIG. 5A-5B. Antibody-based proximity ligation assay (PLA). (FIG. 5A) A pair of antibodies containing DNA oligonucleotide extensions bind the target protein at different epitopes but in proximity to each other. A specific bridge oligonucleotide added in great molar excess rapidly hybridizes to the oligonucleotide extensions from adjacent probes, guiding enzymatic DNA ligation. The ligated DNA sequence is then amplified using real-time PCR and detected. (FIG. 5B) Probes that fail to bind a target molecule and are not in proximity hybridize to one bridge oligonucleotide each, rendering them unable to undergo ligation.

FIG. 6. Correlation Between Probe-Affinity and Assay Sensitivity. The proportion of target proteins bound by a pair of proximity probes at equilibrium can be estimated if the concentration of reagents and the Kd for the interactions are known. By taking into account the background signal observed in the absence of target proteins, these calculations provide estimates of signal over background ratios for various target concentrations, representing theoretical standard curves. The background was empirically measured by varying the concentration of two ligatable oligonucleotide [(B)-3′ and (B)-5′) in 5 μl] incubations, ligated and amplified with sequence-system B. As expected, increasing the concentration of one of the probes five times resulted in a 5-fold increase (4.57±0.62) in background, whereas a 5-fold increase of both probes yielded an ≈25-fold higher background (23.4±3.2-fold). In this figure are estimated standard curves, assuming probe-target interactions with the indicated dissociation constants. These estimates are compared with experimental results from detection of PDGF-BB, thrombin, and insulin. The PDGF-BB aptamers have a reported affinity of 129±11 pM (8), whereas the thrombin aptamers are ≈1 nM (9, 10). The PDGF-BB and thrombin data using SELEX aptamers are from Fredriksson et al. (2). Proximity ligation signals increase linearly with increasing target up to a point where the probability of each target molecule being bound by two probes decreases. This point depends on the affinity of the particular probes used and their concentration. Also included are data generated by using two anti-insulin monoclonal antibodies that form a proximity probe pair after covalent succinimidyl 4-[p-maleimidophenyl]butyrate coupling of oligonucleotides directly to the antibodies (Kd ≈10 nM). The proximity ligation assay for insulin has a sensitivity of 30 pM in 1-μl samples, whereas the detection limit using these antibodies in a 25-μl ELISA is 6 pM (standard assay; Mercodia, Uppsala, Sweden) or 0.42 pM (ultrasensitive assay, Mercodia). The PDGF-BB experimental data closely match the 125 pM theoretical standard curve, whereas the thrombin and insulin data fit the expected results for curves calculated for reagents with a Kd of 0.4 and 2.5 nM, respectively. Probe affinities and assay performance are thus strongly correlated, demonstrating that proximity ligation reactions can also be used to estimate affinities of biomolecular interactions. Moreover, the method could be used to characterize inhibitors of protein-protein interactions [figure from: Gullberg 2003].

FIG. 7. Molecular-inversion probe (MIP). MIP genotyping uses circularizable probes with 5′ and 3′ ends that anneal upstream and downstream of the SNP site leaving a 1 by gap (genomic DNA is shown in blue). Polymerase extension with dNTPs and a non-strand-displacing polymerase is used to fill in the gap. Ligation seals the nick, and exonuclease I (which has 3′ exonuclease activity) is used to remove excess unannealed and unligated circular probes. Finally, the circularized probe is released through treatment with UDG and Nth at a uracil-containing consensus sequence, and the resultant product is PCR-amplified using common primers to ‘built-in’ sites on the circular probe. The orientation of the primers ensures that only circularized probes will be amplified. The resultant product is hybridized and read out on an array of universal-capture probes. [from Fan, Chee & Gunderson. Highly parallel genomic assays. Nature Reviews Genetics 7, 632-644 (August 2006)]

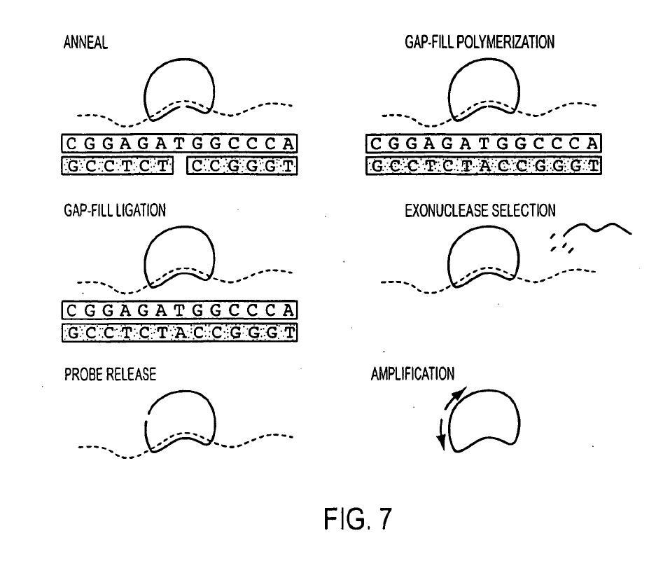

FIG. 8. shows a variety of practical applications of the Tus-Ter binding interaction. Clockwise from top left: DNA-directed immobilization: ZipCoding enables self-assembly on DNA chips, beads, etc.; Molecular velcro: mixed Avidity Body for increased specificity and affinity; Modeling protein complexes: to test pairs of scFv-Tus on oligonucleotide framework; Protein quantitation: Tus:Ter-based PLA does not require chemical conjugation of ZipCodes to mAb. Other refinements will improve specificity; Molecular LEGOs™: enables unique method for assembling protein fusion molecules into pathways and onto solid phase.

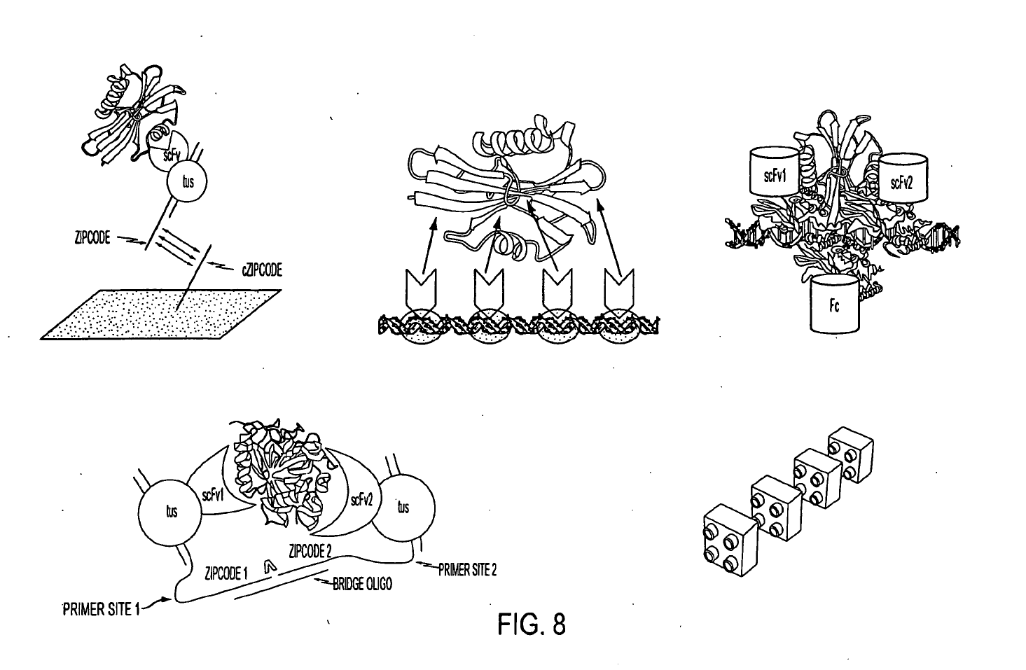

### DETAILED DESCRIPTION OF THE INVENTION

The inventors have developed a web of interrelated methods and products centered on the interaction of Tus protein and the Ter DNA element. This interaction is very strong and permits the conversion of a host of nucleic acid manipulation and detection techniques into techniques for protein manipulation and detection.

The Tus-Ter interaction can be used to make polymers that comprise subunits which are complexes of protein and nucleic acid. The nucleic acid forms the backbone structure of the polymer. The protein portions are fusion proteins (also called hybrid proteins or chimeric proteins) in which one of the fused portions is a Tus protein. The other one or more polypeptides in the fusion protein can be any desired polypeptide. The nucleic acid molecule comprises Ter sites to bind the fusion proteins to the nucleic acid. Typically each Ter site binds a single Tus-containing fusion protein. The monomers in this polymer can be considered a nucleic acid segment with a bound fusion protein. The polymers can be either homopolymers or heteropolymers. The polymers can be block co-polymers, graft co-polymers, or random copolymers.

The polymers may be formed, for example, by attaching a plurality of fusion proteins to a single nucleic acid molecule. Alternatively, the polymers may be formed by attaching a plurality of fusion proteins to a plurality of nucleic acid molecules and subsequently joining the nucleic acid molecules. The nucleic acid molecules may comprise nucleotide analogues which resist nuclease degradation, as well as analogues which stiffen the nucleic acid backbone. Locked nucleotide analogues can be used in this regard. See Semeonov and Nikiforov, Nucleic Acids Research 2002, vol. 30, e91. Ordering of the fusion proteins can be achieved for example using sequential ligation reactions. Alternatively, specific restriction endonuclease sticky ends on nucleic acid molecules can provide sufficient information to specify order of monomers in a polymer. Other means for achieving ordered ligation can be used.

The nucleic acid molecule in the polymer can be completely double stranded or may comprise regions of double strandedness interspersed with regions of single strandedness or nicked double strands. The pattern of single and double stranded bonds may be used to obtain a desired three-dimensional conformation of Tus-containing fusion proteins. Single stranded regions typically provide more flexibility to a polymer than double stranded regions. Double strands are typically more rigid. Nicks can be introduced into a double-stranded backboned polymer using enzymes such as nickases, for example. Alternatively, single stranded nicks may be made between two fragments using a single-stranded ligation reaction. One such reaction employs T4 RNA ligase. Another alternative employs restriction endonuclease digestion of hemimethylated or hemithiolated DNA to make single stranded nicks. Synthetic nucleotide analogues may be used in the single stranded addressing sequences. However, nucleotide analogues will typically not be used in the Ter site itself, in restriction endonuclease sites, and in nickase sites, in order to ensure appropriate binding of proteins. Synthetic nucleotide analogues may be used in order to introduce desired properties into a nucleic acid molecule. These include without limitation resistance to nuclease digestion, increased polymer rigidity, labels, reactive moieties, etc.

The polypeptide(s) that is fused to the Tus protein, may be, for example, any desired protein, antigen, epitope, tag sequence, enzyme, or any binding polypeptide that binds a target molecule. One polypeptide fused to Tus may be an scFv fragment. Optionally, at least one polypeptide may be an scFv fragment and at least one polypeptide may be an Fc domain. Alternatively, at least two polypeptides are scFv fragments and at least one polypeptide is an Fc domain. Two scFv fragments in a polymer or oligomer can be identical or non-identical (distinct); they may bind to the same epitope, different epitopes, or different antigens. Polymers which employ scFv fragments in the fusion proteins can be used to model, mimic, or recapitulate a native antibody structure. The Fc domain may be from any isotype of antibody, such as IgGA, IgGD, IgGE, IgG1, IgG2, IgG3, IgM, etc. The polypeptides need not, however, be scFv. Other polypeptides which can be used, include ligands, receptors, pro-drugs, fluorescent proteins, enzymes. In one embodiment, a plurality of enzymes are joined together on a nucleic acid backbone as Tus fusion proteins; the enzymes participate in a metabolic or biosynthetic pathway. In one particular embodiment, enzymes are ordered in the polymer spatially corresponding to the enzymes' function temporally in the metabolic or biosynthetic pathway. Thus the product of a first enzymatic conversion can “pass” to a second enzyme where it is a reactant, and the product of conversion by the second enzyme can “pass” to a third enzyme where it is a reactant. “Passing” is used here to denote diffusion over a short distance from one enzyme to another.

Polymers can be made by ligating a plurality of monomers (complexes of DNA and nucleic acids) to each other using a DNA ligase enzyme. Each monomer may comprise a non-covalent complex of a fusion protein (comprising a Tus protein and a polypeptide), and a nucleic acid molecule (comprising a Ter site). In some cases, it may be desirable that some nucleic acid molecules contain no fusion protein bound to them. The nucleic acid molecules in the monomers may have 5′ and 3′ sticky ends. The 5′ and 3′ sticky ends of the nucleic acid molecules may be identical or distinct. Distinct ends may be used to facilitate the ordered assembly of monomers. As mentioned above, the polypeptides in the fusion proteins may be enzymes in a metabolic or biosynthetic pathway. The enzymes may be spatially ordered in the polymer corresponding to the temporal sequence of the enzymes' function in the enzymatic pathway. Polymers may function in solution or they may themselves be tethered to a substratum. The substratum may be, for example, a bead, an array, a chromatography matrix. Use of a substratum permits the ready separation of enzymes and products. Any means known in the art for attaching a nucleic acid or a protein to a solid support may be used. These include covalent and non-covalent attachments, for example, nucleic acid hybridization, biotin-avidin, chemical coupling.

Polymers can also be made by mixing proteins with a nucleic acid comprising more than one Ter sites. For example, a first and a second fusion protein can be mixed with a nucleic acid molecule which is pre-bound to a third fusion protein. The nucleic acid molecule comprises at least three Ter sites. Each fusion protein comprises a Tus protein and a polypeptide. The first and second fusion proteins comprise a first and second scFv fragment as the polypeptide. The third fusion protein comprises an Fc fragment of an immunoglobulin molecule as the polypeptide. In other embodiments, the fusion proteins comprise any polypeptide, not necessarily an scFv fragment or an Fc fragment.

Monomer complexes and libraries of such monomer complexes can be used inter alia to attach to a substratum, such as an oligonucleotide array. The library is a composition comprising a plurality of diverse protein-DNA complexes. Each complex comprises a Tus fusion protein and a nucleic acid molecule. The fusion protein may comprise a Tus protein and an scFv fragment. Alternatively, the Tus protein is fused to other types of polypeptides, particularly binding polypeptides, and more particularly antigen-binding polypeptides. Binding polypeptides need not be antibody molecules or antibody related or derived. They may be enzymes, ligands, receptors, substrates, or inhibitors, for example. A first portion of the nucleic acid molecule is double stranded and a second portion of the nucleic acid molecule is single stranded. The first portion comprises a Ter sequence (for binding to the fusion protein) and the second portion comprises an addressing sequence (for hybridizing to a nucleic acid on a substratum). Typically each addressing sequence on a nucleic acid molecule is complexed with a fusion protein comprising a unique binding polypeptide, i.e., there is a correspondence (typically a 1:1 correspondence) between a binding polypeptide and an address. One can conceive of situations where one may want to place the same binding polypeptide at two locations on or on two members of an array thus using a ratio of less than 1:1. One can also conceive of situations wherein two different binding polypeptides would be attached to the same location, thus using a ratio of more than 1:1. Even these variations from 1:1 are considered herein as a unique relationship because there is a corresponding relationship between the address and the binding polypeptide.

Libraries of monomer complexes may be packaged in a container as such, for example as a liquid or solid, frozen or lyophilized. The library may be a single composition or a divided composition. The library may be already attached to one or more substrata or not yet attached. The substrata may be provided together with or separately from the library. The substratum may have geographically located single stranded probes, each of which comprise a sequence of at least 6 nucleotides which is complementary to an addressing sequence in the nucleic acid molecules of the monomer complexes. Such a substratum is frequently referred to as an array or a chip. These are available commercially. Alternatively beads or nanoparticles can be used as substrata. Such substrata have a uniquely identifiable or detectable label. For example, each bead may be labeled with a unique barcode, dye, dye concentration, or radiolabel. Such substrata form a suspended array rather than a geographically located array. Alternatively the monomer complexes may be used for binding to moieties other than substrata, such as fluorescent labels. Such complexes may be used in a homogeneous phase reaction. In these situations, as in the case of a substratum, the complexes are attached to another moiety using hybridization of single strand addresses. As discussed elsewhere, “unique” as used here does not require a strict one-to-one relationship. Rather a correspondence or relationship between two elements is intended.

Addressing sequences that are present in the Tus-Ter complexes may be at least 6, at least 8, at least 12, at least 14, at least 16, at least 18, at least 20, at least 22, at least 24, at least 25, at least 26, at least 28, or at least 30 nucleotides in length. Specificity may depend on the complexity of mixtures of sequences and the conditions under which hybridization of single strands occurs. Similarly, the complements of the addressing sequences that are found, for example, on an oligonucleotide array, may be at least 6, at least 8, at least 12, at least 14, at least 16, at least 18, at least 20, at least 22, at least 24, at least 25, at least 26, at least 28, or at least 30 nucleotides in length.

In a geographically arrayed library of binding polypeptides or antigen-binding polypeptides, such as scFv fragments, each binding polypeptide is typically tethered to the array using non-covalent binding of a Tus protein to a Ter sequence. Each binding polypeptide is fused to a Tus protein, forming a fusion protein. Each Ter sequence is within a nucleic acid molecule comprising double and single stranded portions. The single stranded portions comprise an addressing sequence and the double stranded portions comprise the Ter sequence. The addressing sequence is complementary to a single stranded probe which is attached to a substratum, thus the addressing sequence can hybridize to the probe, thereby accomplishing the arraying of a library of binding polypeptides. The single stranded probes may be attached to the substratum by means of non-covalent interactions (such as biotin-streptavidin interactions) or by means of covalent bonds (as made, for example, using photolithography).

Target molecules can be measured using two distinct target-binding polypeptides, such as scFv fragments. A first and a second binding polypeptide are mixed with a target molecule to be measured, forming a mixture. Each binding polypeptide is part of a fusion protein with a Tus protein and the Tus protein is bound to a DNA molecule which comprises a double-stranded portion and a single-stranded portion. The double-stranded portion comprises a Ter sequence, and the single stranded portion comprises a tag sequence which is unique to (or corresponds to) the binding polypeptide. A bridging oligonucleotide is added to the mixture under conditions in which complementary DNA single strands form double strands. The bridging oligonucleotide comprises a first and a second portion. The first portion is complementary to the tag sequence of the first binding polypeptide and the second portion is complementary to the tag sequence of the second binding polypeptide. The first and the second portion of the bridging oligonucleotide are separated by 0 to 6 nucleotides. DNA ligase is added to the mixture; the ligase joins 5′ and 3′ ends of nicked double-stranded DNA molecules. An assay is performed to determine amount in the mixture of an analyte DNA strand comprising both the tag sequence of the first antigen-binding polypeptide and the tag sequence of the second antigen-binding polypeptide. The amount of the analyte DNA molecule is related to the amount of the target antigen. If the first and the second portions of the bridging oligonucleotides are separated by 1 to 6 nucleotides they form a gap. The gap can optionally be filled in by addition of a DNA polymerase and deoxynucleotides to the mixture prior to adding the DNA ligase. The DNA polymerase fills in single-stranded gaps of less than 7 nucleotides in a double-stranded DNA molecule. The use of a gap and fill-in reaction are optional, but may improve the specificity of the analysis. If there is no gap, i.e., the first and second portions of the bridging oligonucleotides are separated by 0 nucleotides, then no fill-in reaction need be performed. In order to facilitate detection and quantitation of the analyte DNA molecule, it can be amplified using as non-limiting examples, a polymerase chain reaction, rolling circle reaction, and ligase chain reaction. Any means of detection of the analyte can be used. Another optional step is to use an exonuclease to remove non-ligated molecules after the ligation reaction. This typically reduces background noise in the detection reactions.

Enzymes can be attached to a substratum, individually, in tandem arrays, in mixtures, or in ordered mixtures. The attachment is done via a nucleic acid intermediary. The nucleic acid molecule is attached to the substratum by means of covalent or non-covalent coupling. The coupling may, for example, be via biotin-streptavidin interactions. The nucleic acid molecule comprises at least one Ter sequence and may be non-covalently complexed with one or more fusion proteins that comprise a Tus protein and an enzyme. If the nucleic acid molecule is not already complexed with one or more Tus fusion protein(s), then subsequent to its attachment to the substratum one or more fusion protein(s) can be attached to the nucleic acid molecule via the Tus-Ter binding interaction. The fusion proteins may optionally comprise enzymes that function in an enzymatic, e.g., metabolic, biosynthetic, or catabolic pathway. Optionally the plurality of fusion proteins are in a predetermined spatial order, corresponding to the sequence in which the enzymes function temporally in the enzymatic pathway. Examples of substrata which may be used are chips, chromatography matrices, liposomes, and beads.

Arrayed libraries of diverse protein-DNA complexes can be made by mixing together a substratum comprising one or more single stranded probes and a library of diverse protein-DNA complexes. Each protein-DNA complex comprises a fusion protein and a nucleic acid molecule. The fusion protein comprises a Tus protein and a binding polypeptide. A first portion of the nucleic acid molecule is double stranded and a second portion of the nucleic acid molecule is single stranded. The first portion comprises a Ter sequence and the second portion comprises an addressing sequence. Each addressing sequence is complexed with a fusion protein comprising a unique or corresponding binding polypeptide. There is a correspondence between the addressing sequence and the binding polypeptide. The single-stranded probes each comprise a sequence of at least 6 nucleotides which is complementary to an addressing sequence in the nucleic acid molecules of the protein-DNA complexes. Upon mixing, the protein-DNA complexes bind to single stranded probes having complementary sequences by Watson-Crick hybridization. Binding polypeptides which may be used include scFv fragments, ligands, receptors, enzyme substrates, substrate analogues, enzymes, and enzyme inhibitors. Arrayed libraries can be arrayed on geographical arrays on substrata including silicon chips or glass slides, or suspended arrays on substrata including beads or chromatography matrices.

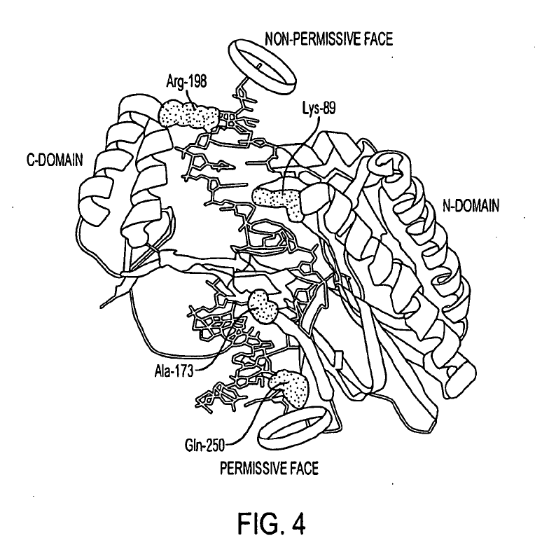

The DNA replication termination protein Tus blocks the progress of the replisome in the final stages of chromosomal replication in E. coli and related bacterial species (Mulcair 2006, Torigoe 2005, Mizuta 2003, Neylon 2000, Duggin 1995, Duggin 1999, Skokotos 1994, Skakotos 1995). The Tus protein binds as a monomer to Ter sites situated in the terminus region of the bacterial chromosome in such a way as to form a replication fork trap (FIG. 4). The progress of a fork is halted when traveling in one direction (from the non-permissive face of the complex) but not the other (the permissive face). Replication forks traveling in both directions are therefore able to enter the terminus region but not leave it. The Tus:TerB interaction is one of the strongest among protein-ligand interactions and is the strongest known DNA-protein interaction involving a monomeric DNA-binding protein. The native Tus protein binds to the TerB site, for example, with an equilibrium dissociation constant (K_{D}) of 3.4×10⁻¹³ M in 150 mM potassium glutamate, pH 7.5.

The Tus-TerB complex is very stable, with a half-life of 550 min, a dissociation rate constant of 2.1×10⁻⁵ s⁻¹, and an association rate constant of 1.4×108 M⁻¹ s⁻¹. Similar measurements of Tus protein binding to the TerR2 site of the plasmid R6K showed an affinity 30-fold lower than the Tus-TerB interaction. This difference was due primarily to a more rapid dissociation of the Tus-TerR2 complex. Using standard chemical modification techniques, the DNA-protein contacts of the Tus-TerB interaction were examined. Extensive contacts between the Tus protein and the TerB sequence were observed in the highly conserved 11 base-pair “core” sequence common to all identified Ter sites. The consensus sequence of E. coli Ter sites A-J and R6K TerR1 and TerR2 is AGNATGTTGTAACTAA (SEQ ID NO: 7). Permissible substitutions (indicated in parenthesis) may be made at positions 1 (N), 3 (G), 4 (N), 13 (T), 14 (G), and 16 (N).

The crystal structure of the Tus:Ter complex (Kamada 1996) indicates that the core DNA-binding domain of the protein consists of two pairs of antiparallel α-strands that lie in the major groove of the DNA. Kamada et al. (Kamada 1996) identified 14 residues that make sequence-specific contacts to the Ter DNA. Ten of these lie within the core DNA-binding domain and four lie outside it.

Bacterial Tus proteins and Ter sequences may be used from species of bacteria other than E. coli, particularly other gram negative bacteria, particularly from other enterobacteria, particularly form other strains of E. coli. A Ter sequence comprising a core of 11, 12, 13, or 14 nucleotides may be used, for example. Other E. coli Ter sequences may be used including any of Ter sequences A-J, and R6K plasmid Ter sequences TerR1 and TerR2. A Ter consensus sequence is shown in SEQ ID NO: 7 and any sequence conforming to this consensus may also be used.

Desirably, variants of Tus protein and/or Ter sequences will retain a Kd of less than 10⁻¹², less than 10⁻¹¹, or less than 10⁻¹⁰. Variants will typically vary from SEQ ID NO: 5 or 7 in less than 10% of the amino acid or nucleotide residues, in less than 5% of the amino acid or nucleotide residues, in less than 2% of the amino acid or nucleotide residues, or in less than 1% of the amino acid or nucleotide residues.

Tus:Ter interaction and application in the development of self-assembling protein arrays. The high-throughput deposition of recombinant proteins on chips, beads or biosensor devices is greatly facilitated by self-assembly. DNA-directed immobilization (DDI) via conjugation of proteins to an oligonucleotide is well suited for this purpose. DDI of proteins has been estimated to be 100-fold more economical in the use of purified protein material compared to direct spotting of proteins on substrata [Nedved 1994]. This advantage would become even more significant if lower protein concentrations and smaller spot sizes could be used. The current technology for DNA arrays is in the 40-μm range for spot sizes, but soft lithography techniques can create arrays of 40-nm dimensions. Such arrays can be interlaced with grids of 2- and 3-D DNA assemblies as described by Seeman [2003]. These advances in DNA arrays allow the precise positioning of arrays of protein clusters or even single protein molecules in a process of self assembly. DDI is at least as effective as current spotting methods and provides robust, high functional scFv arrays.

In one aspect, one can isolate the Tus fusion proteins from an Escherichia coli lysate and attach them to a DNA addressing sequence (or ZipCode). Tus fusion protein binding to endogenous Ter sites in the E. coli chromosome during isolation and/or purification can be overcome by the massive over-expression of the Tus fusion protein. Expression of Tus fusion proteins can be accomplished, if desired, in strains that are Ter deficient.

High throughput antibody discovery. The term proteomics has been applied to efforts to describe parallel processing systems that permit functional analysis of most or all proteins encoded by an organism. Currently the rate of proteomic analysis is not comparable to that which can be achieved by mRNA profiling approaches. However, many of the techniques disclosed here permit mRNA profiling approaches to be subverted for protein profiling.

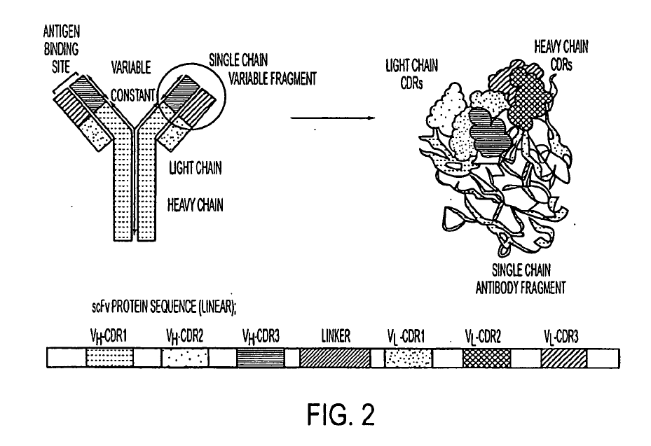

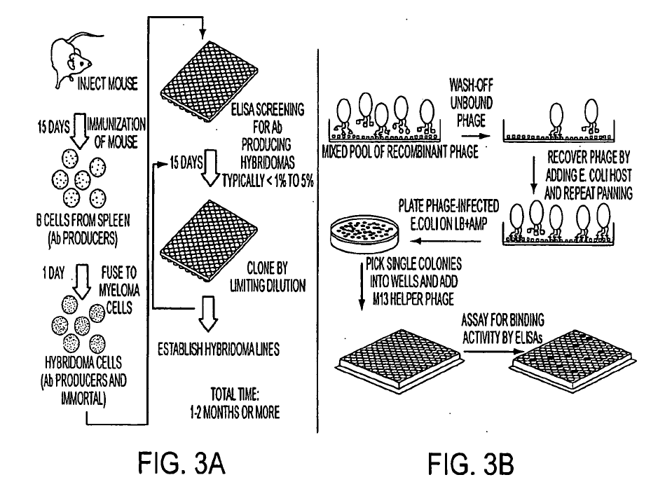

Antibodies, and particularly monoclonal antibodies (mAbs) are prototypic affinity reagents for identification and quantitation of proteins in a sample. FIG. 2 illustrates a generic antibody structure. The development of hybridoma technology (FIG. 3A) represented a revolutionary approach for the selection of mAbs with desired affinities and specificities for a target antigen. Although this approach has been used repeatedly and successfully for generating antibodies, it is far too costly and tedious to be used for the generation of a proteomic affinity set. A second method used for generating mAbs of high quality is phage display (FIG. 3B, and Lee 2004; Sheets 1998). In this method a library of single chain, variable fragment (scFv) antibodies are displayed on the surface of M13 bacteriophage gpIII as genetic fusions to the gpIII protein and used in ‘biopanning’ procedures against an antigen of interest. Although phage display offers efficiencies and cost savings relative to hybridoma technology, the need for several biopanning, wash, plating, and ELISA steps in the current manifestation does not present a compelling approach for making tens of thousands of antibodies. An automated yeast two-hybrid approach for selecting scFv against target antigens could satisfy such needs (R. Buckholz, et al., Automation of Yeast Two-hybrid Screening 1999, JMMB Communication 1:135-140.)

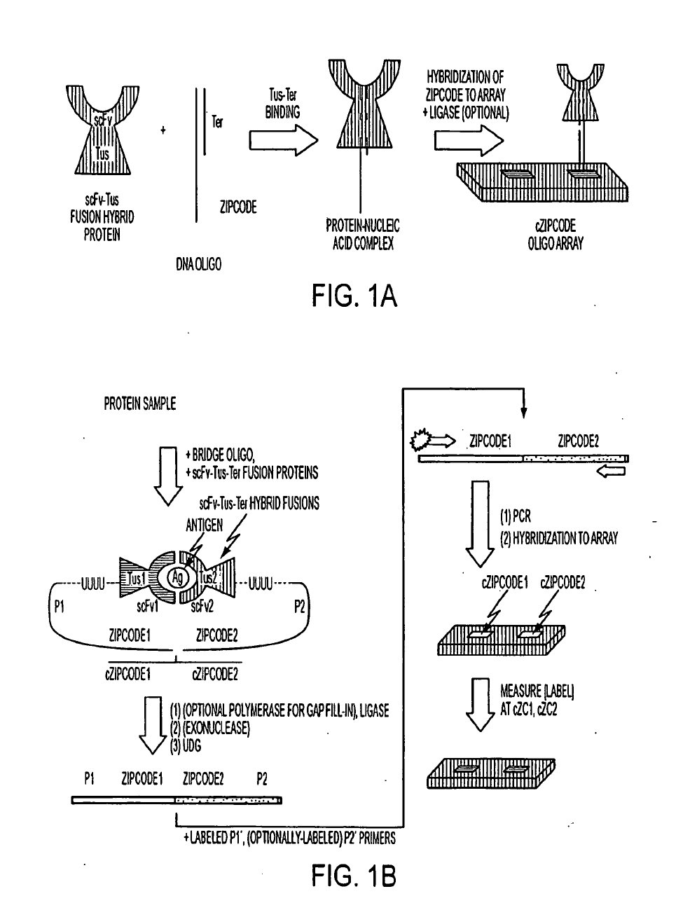

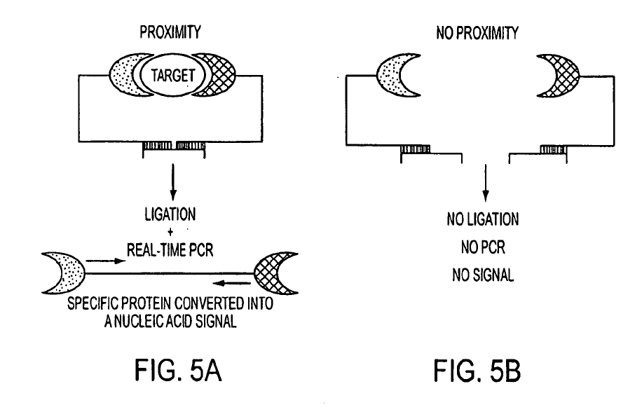

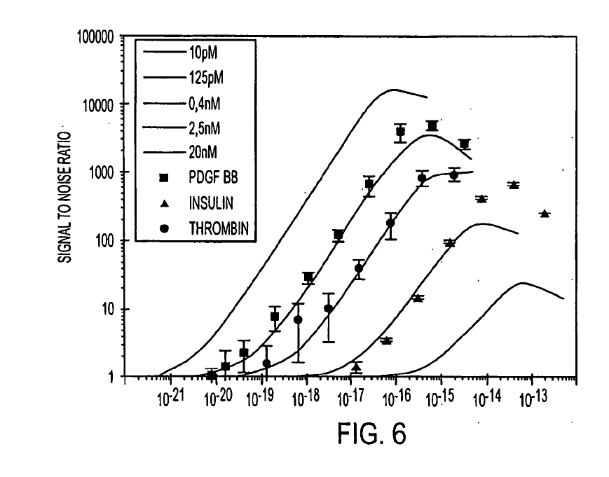

Proximity Ligation Assay. PLA is a recently developed strategy for protein analysis in which antibody-based detection of a target protein via a DNA ligation reaction of oligonucleotides linked to the antibodies results in the formation of an amplifiable DNA strand suitable for analysis [Dahl 2005, Fredriksson 2002, Gullberg 2003, Gullberg 2004, Gustafsdottir 2007, Jarvius 2007, Landegren 2004 Schallmeiner 2007, Soderberg 2007, Zhu 2006]. In PLA, pairs of proteins (in this case antibodies) containing oligonucleotide extensions are designed to bind pair-wise to a target protein and to form amplifiable tag sequences by ligation when brought in proximity (see FIGS. 1B and 5). Excellent sensitivity is ensured by the great increase in reactivity of ligatable ends on coincident target binding through increased relative concentration in combination with amplified DNA detection by real-time PCR, enabling the measurement of very few ligation products. PLAs can also be performed by using a solid phase format and, due to its proximity-dependent signal, it has displayed higher sensitivity than another DNA-based protein detection assay, immunoPCR [Adler 2005, Barletta 2006]. FIG. 6 provides an example of the use of PLA for quantitating target protein levels.

PLA is suitable for automation in high-throughput applications because it can be designed to be homogeneous, i.e., no washing steps are involved, and the procedure requires only the sequential additions to the incubation mixture of (1) the sample and (2) a ligation-PCR mixture. The high sensitivity of PLA allows 1-μl sample aliquots to be monitored, minimizing sample consumption and thus enabling analysis of samples available only in very small amounts that would not be measurable by traditional techniques. Also, 1,000-fold less antibody is used per assay compared to standard ELISAs, and because all assays perform favorably at similar reagent concentrations, new assays do not require extensive optimization. The precision of proximity ligation is currently at the level of real-time PCR detection, but improved quantitative detection strategies for nucleic acids may offer a further increase in precision [Soderberg 2006]. PLA is ideal for multiplexed detection, which is a goal for many technologies under development, especially antibody-based microarrays. As more detection reactions are performed in parallel, the issue of antibody cross-reactivity becomes an increasing problem limiting scalability. PLA offers a possible solution to this problem if unique ligation junctions are used for each cognate proximity probe pair. Finally, by including a unique and amplifiable ZipCode sequence within the oligonucleotide attached to each different antibody, parallel analyses may be possible with PLA, allowing standard oligonucleotide capture arrays to be used for absolute or relative measurements of large sets of different proteins.

Molecular Inversion Probe (MIP) Technology [Hardenbol 2003, Moorhead 2006, Wang 2005]. One PLA-related technology is known as Molecular Inversion Probes (FIG. 77). MIPs have two specific homology sequences that leave a 1 by gap when hybridized to an otherwise complementary sequence [Wang 2005]. MIPs also contain specific tag sequences that are ultimately bound to a DNA microarray. In addition to these elements that are specific to each probe, there are two PCR primers that are common to all probes. These primers face away from each other and therefore cannot facilitate amplification. After the probes are hybridized, the nucleotide is added to the tube. The gap is filled-in in the presence of the appropriate nucleotide. A unimolecular ligation event is then catalyzed. After eliminating the single stranded portions of the probes with exonucleases, PCRs using the common primers that now face each other is performed in the tube. In addition to signal amplification a fluorescent label is introduced by a PCR primer. The reaction is then hybridized onto a tag array. As many as 22,000 single nucleotide polymorphism (SNP) markers from an individual sample can be interrogated. The MIP technology has several features that convey advantages for this application over other methods using oligonucleotide arrays. In the assay, a high degree of specificity is achieved through a combination of the unique unimolecular probe design and selective enzymology which also allows the technology to be very highly multiplexed. The tag-based read-out array also conveys distinct advantages. By avoiding the use of genomic sequences to separate the signals on the array, cross hybridization levels among the different probes can be kept at a very low level, allowing signals to be quantitated with high precision.

Double stranded DNA behaves as a relatively rigid molecular rod. A nick, or single-stranded break in the backbone allows the molecule to rotate around the other strand, thereby introducing flexibility into the structure. The nick can be created by ligation of a single phosphate or by using a nickase enzyme. The introduction of flexibility or rotation in a nucleic acid backbone, which is especially useful in trying to optimize the spatial relationship of protein subunits (Tus-hybrids) attached to the DNA(containing Ter).

T4 DNA ligase requires double stranded DNA with at least one 5′ phosphate adjacent to a 3′ hydroxyl group. Ligating a double stranded DNA having only a single phosphate and adjacent hydroxyl will create a nicked molecule, which confers flexibility to the structure. Conversely, the nucleic acid backbone can be further stiffened using modified nucleotides, for example, locked nucleotides (LNAs).

Nickase enzymes are similar to restriction enzymes except that they recognize an assymetric DNA sequence and nick one (but not both) strands of the DNA. Several of the nicking endonucleases are commercially available, including Nb.BbvCI, Nb.BsmI, Nb.BsrDI, Nb.BtsI, Nt.AlwI, Nt.BbvCI, Nt.BspQI, Nt.BstNBI, Nt.CviPII. New England Biolabs, Beverly, Mass.

Tus fusions can be used to place binding polypeptides, such as scFv and other functionalities, including Fc regions, GFP, βgal, HRP, luciferase, etc, onto a DNA molecule to model an IgG molecule or a modified IgG molecule. This permits one to conveniently generate pseudo-IgG-like molecules for testing in in vivo or in vitro assays. One can use T4 DNA ligase and/or nickase enzymes to vary the spatial conformation of the Tus fusions. When suitable binding scFvs have been identified in such assays, the CDRs can be cloned from the scFv constructs and used to reconstitute full antibody (for example, IgG) molecules. In one embodiment, two different suitable binding scFvs are found to bind effectively to a target antigen as part of a pseudo-IgG-like molecule and introduction of CDRs from two different suitable binding scFvs in the respective Fab regions of a full antibody molecule generates a heteroantibody that not only results in high affinity binding but also confers high levels of specificity for target antigen.

Multiple functionalities (in this example, a trimeric scFv chimera) can be used to differentiate the chimeric tus-fusion-DNA molecules binding to different cell types. In this example, cell type A displays 1 of 3 antigens on the cell surface, cell type B displays another of the three antigens on the surface, whereas cell type C displays all 3 antigens. The binding molecules would be quickly tested to derive binding affinities that would enable the chimeric trimer to bind with higher affinity to cell type C than to cell types A or B.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLES

In the following Specific Examples and Alternative Examples, (1) TerB refers to the 21-nt double-stranded DNA TerB sequence 5′-ATAAGTATGTTGTAACTAAAG-3′ (SEQ ID NO: 1), and where indicated, a short single-strand ZipCode DNA sequence extended from one or both (i.e., Watson and/or Crick) strands of the TerB oligonucleotides; (2) ZipCode and cZipCode represent 20-30 nt complementary sequences of single-stranded DNA; (3) for the sake of brevity and because of our prior experience, the examples described in the following Specific Examples use Luminex™ beads as the solid support, although other formats of oligonucleotide arrays (as non-limiting e.g., Affymetrix™ and Nimblegen™), can extend the analysis by incorporating additional ZipCodes and cZipCodes in multiplex reactions. In the examples we use scFv:antigen as the polypeptide binding interaction.

It should be noted that there are many examples where the interacting pair may not involve scFv moieties. Rather, one could imagine quite easily where the techniques described are used to assay a target molecule using a non-scFv affinity reagent, provided the affinity reagent can be coupled to a DNA molecule. Thus pairs of affinity reagents can be identical or non-identical, forming homooligomers or heterooligomers. One member of a pair may be an scFv and one member may be a receptor or ligand that binds to the same or a different epitope or antigen, for example.

The interacting pairs do not need to be proteins. It is possible to use the described technology to evaluate ligand binding to other ligands and to proteins. It is possible to use the described technology to evaluate the interactome.

DNA binding proteins other than Tus that recognize specific sequences or specific morphologies of DNA are known in the art, and such combinations of proteins and their cognate sequences can be used in this invention provided that the Kd of their interaction is less than 10¹⁰. Examples of such DNA binding proteins include recA, DNA restriction enzymes, DNA methylation enzymes, DNA ligases, ruvA, ruvB, ruvC, or other enzymes that recognize DNA mismatchs, DNA repair enzymes, helicases, polymerases, transcription factors. A thiolated ATP (gamma S thiol ADP) molecule can be used to bind a recA protein irreversibly to DNA.

The methods can be adapted to RNA-binding proteins, for example tRNA synthetases, capping enzymes, RNA polymerases.

The methods can be adapted to protein-binding proteins. The fusion-binding can be to a protein or polymer of a peptide or peptide repeating units. Finally, there are several further examples of the described technology including: (a) PLA with the anti-phosphotyrosine monoclonal antibody PY20 to monitor phosphorylation of proteins; (b) increasing scFv avidity by use of a polyTer sequence of catenated Ter sequences; different Ter-Tus may allow us to generate enzyme pathway fusions for use as a type of dendritic resin in column chromatography; (c) Tus can serve as the DNA-binding partner in a protein-protein interaction trap or act as an endogenous repressor in an in vivo or in vitro system; (d) the binding of the fusions can be transient, or irreversible. If irreversible it can be by chemical or other means, for example UV irradiation.

Reagents can be developed using Tus-Ter for increased avidity. Such reagents may employ Tus fusion proteins that comprise identical or different target binding polypeptides. For example, a nucleic acid backbone comprising a plurality of Ter sequences can be used to attach a plurality of Tus fusion molecules. If the fusion proteins comprise the same binding polypeptide, then the avidity may be increased by a mechanism in which a first binding polypeptide in the neighborhood of a second polypeptide can bind to a target molecule when it is released from the second polypeptide. Thus the greater number of binding moieties increases the amount of time that a target molecule is bound to the Tus-Ter complex rather than unbound. Conversely, if the fusion proteins comprises distinct binding polypeptides that bind to different portions of a target molecule, for example, to two epitopes of a single antigen, then avidity will be increased because a single target molecule can be bound simultaneously by two binding polypeptides in a single Tus-Ter complex. Thus both heterogeneous and homogeneous binding polypeptides can be bound to a single nucleic acid molecule via Tus and Ter to create reagents of increased avidity. Applicants do not intend to be bound by any theory of mechanism of action.

Enzymes involved in a specific metabolic pathway (for example ethanol production) can be catenated in order to create chromatography columns or other substrata that are more efficient at catalytic conversion. The efficiencies of having active enzymes, in solution and in close proximity is that diffusion-limited reactions will proceed much faster. Similarly, we can ligate or hybridize the ZipCodes in an ordered fashion onto a longer DNA fragment to create an ordered array of enzymes. Using branched oligonucleotides, one can construct 3-dimensional lattices of either random or ordered enzymes.

### Example 1

### Quality-Controlling Reagents: Proteins and Oligonucleotide-Coupled Beads

The fusion of a Tus protein with either a binding polypeptide or GFP, as non-limiting examples, can be cloned and purified from E. coli using a T7 expression system [Neylon 2000]. As a non-limiting example, His6 affinity tag can be fused to the amino terminus of the protein. It has been shown that this tag does not alter enzyme activity. It is known that both GFP and scFv proteins can tolerate carboxyl- and amino-terminal fusions. We have already selected and isolated several scFv mAbs that can be expressed in the cytoplasm of E. coli.

(1) Protein cloning and protein purification. Tus can be amplified from E. coli XL1Blue (Stratagene, La Jolla, Calif.) using TusF1 (5′-ATGTTGTAAC TAAAGTGGTT AATAT-3′; SEQ ID NO: 2) and TusR1 (5′-TTAATCTGCA ACATACAGGA GCAGC-3′; SEQ ID NO: 3) primer pair. GFP, as a non-limiting example, can be amplified from the phMGFP Vector (Promega Corp, Madison, Wis.). We can use our own or other scFv as the test antibody. The genes can be cloned into, and expressed from a T7 RNA polymerase pETMCS his6 affinity-tag vector (Stratagene). The process of making fusion constructs is well-known in the art. The cells can be induced with IPTG and allowed to continue growth overnight at either room temperature (RT) or 30° C. Cells can be lysed by sonication and protein purification can use Ni(III), where applicable, and size-exclusion column chromatography. The final fractions containing Tus-containing fusion protein can be exchanged into storage buffer (50 mM Tris-HCl, pH 7.5, 100 mM NaCl, 1 mM DTT, 1 mM EDTA, 20% w/v glycerol), concentrated using a vacuum dialysis apparatus (Schleicher and Schuell), and stored at −80° C. Tus-fusion protein concentrations can be determined from its UV absorption spectrum. (2) TerB and reverse TerB (rTerB), ZipCode and cZipCode design and synthesis. TerB (5′-ZipCode-ATAAGTATGT TGTAACTAAAG-3′ (SEQ ID NO: 1) and 5′-CTTTAGTTAC AACATACTTAT-3′ (SEQ ID NO: 4)) and rTerB (5′-ZipCode-CTTTAGTTA CAACATACTTAT-3′ (SEQ ID NO: 4) and 5′-ATAAGTATGT TGTAACTAAAG-3′ (SEQ ID NO: 1)) can be purchased from commercial sources (IDT). The ZipCodes and cZipCodes can be based on the non-cross-hybridizing sets of oligonucleotide ZipCodes previously used for genotyping on Luminex™ beads [see Taylor 2001]. Attachment of the cZipCode oligonucleotides to Luminex™ beads can use, as a non-limiting example, standard EDC (1-Ethyl-3-[3-dimethylaminopropyl]carbodiimide hydrochloride)-based coupling. Coupling reaction success can be assessed by hybridizing coupled microspheres with a molar excess of fluorescein-labeled oligonucleotide complementary to the cZipCode sequence. Our experience has shown that effective coupling reactions produce microspheres with mean fluorescence intensity (MFI) of 2000-4000 U. Microspheres with MFIs less than 1000 can be replaced.

Alternative examples. (1) Both the scFv and GFP gene clones can be synthetic constructs and can be designed to express well in bacterial cytoplasm. If necessary, other host expression organisms (as a non-limiting examples: insect, adenoviral, Bacillus, E. coli, mammalian and yeast expression systems). (2) Extended hydrophilic linkers of various sizes [for example, as a non-limiting example, of the general type (Gly4Ser)N] can be placed between the Tus and the fusion partner. (3) We can also control expression by changing the inducing regimen [overnight at RT, 30° C. or 37° C., modify the timing and concentration of the IPTG inducing agent, and express the protein in the presence or absence of thioredoxin]. For E. coli expression, proteins can be designed to be secreted into the periplasm, which has been shown in some cases to reduce cytoplasmic aggregation. Both scFvs and GFPs, as non-limiting examples, are at the amino terminus of the fusion hybrid and are known to be efficiently secreted when suitably genetically-fused to a leader peptide. (4) In the example above, a His6 affinity tag is included in the protein fusion to facilitate purification but other affinity tags, as known n the ar can be incorporated into the fusion protein to facilitate purification. (5) Bead fluorescence can be measured using a Luminex™ 100 cytometer equipped with a Luminex™ plate reader and Luminex™ software.

### Example 2

### Parameters for Self-Assembling Protein Arrays

DNA-DNA hybridization of the ZipCode and cZipCode sequence can enable the DNA-directed immobilization and the PLA. The directional nature of Tus replication arrest may be explained by the asymmetry of the Tus:Ter complex. The directional nature of the interaction may cause the fusion hybrid to function more efficiently in one direction. We can test this by binding the Tus hybrid to both TerB and a reverse TerB (rTerB).

Experimental design and expected outcome. (1) GFP-Tus:TerB-ZipCode and GFP-Tus:rTerB-ZipCode binding to cZipCode coupled beads. To test the ZipCode binding of the fusion protein in two orientations, we can bind the His6-GFP-Tus hybrid separately to TerB-ZipCode1 and rTerB-ZipCode1 DNA sequences, and then bind them to the cZipCode1 beads. Protein solutions can be diluted in Tus:TerB binding buffer (50 mM Tris-HCl, 0.1 mM EDTA, 0.1 mM DTT, 0.005% Nonidet P-20, 150 mM KCl, pH 7.5). A 5× excess molar amount of TerB-ZipCode1 (or rTerB-ZipCode1) can be added. The protein can be separated from the free oligonucleotide using, as a non-limiting example, a Ni(III) column. The protein can be eluted from the column and can be added to both cZipCode1-coupled and negative control beads (the negative control bead can be a second, non-complementing cZipCode2-coupled bead). The beads can be washed as described above, and the amount of bound GFP measured either on a spectrometer or Luminex 100. (2) His6-scFv-Tus:TerB-ZipCode1 and His6-scFv-Tus:rTerB-ZipCode1 binding to cZipCode1 and cZipCode2 beads. Using conditions in the previous Specific Example (2.1), we can test the usefulness of scFv-Tus hybrids in two orientations. We can use, as a non-limiting example, an anti-GCN scFv as the test antibody. We can use binding to a fluorescein-labeled GCN peptide as the positive-control test ligand.

Additional non-limiting examples. (1) We can test the Tus-fusion:TerB-ZipCode1 binding to the cZipCode1 on the bead by labeling the cZipCode1 with fluorescein, hybridize this labeled oligonucleotide to His6-Tus:TerB-ZipCode1, and then use, as a non-limiting example, a Ni(III) column to purify the protein complex and determine if cZipCode1 is hybridizing to ZipCode1. We can add, as a non-limiting example, a 10 unit abasic deoxynucleotide spacer between TerB and the ZipCode to lengthen the distance between the binding subunits in the complex. If necessary, we can also use a series of longer ZipCodes. As an optional step, we can use the ability of T4 DNA ligase to covalently bind the Tus:TerB complex to either a bead or cZipCoded array. There may be an impart of a stability advantage to have the complex bound in this manner. (2) We can use GFP-Tus:TerB fused to a non-complementing ZipCode as a negative control. If non-specific interaction is a problem, we can test several blocking agents (as a non-limiting example, e.g., non-fat dry milk powder, BSA, tRNA, etc). We can test each component of the system both separately and together to test conditions associated with oligonucleotide hybridization. Longer hybridization probes can be used to enable hybridization. Tus:TerB interaction occurs in a buffer that is compatible with DNA-DNA hybridization. Note also for Specific Example 4 that by having ZipCode tags on both ends of TerB we will be able to simultaneously perform both solid-phase hybridization and PLA.

### Example 3

### Storage Parameters for the Fusion Library for Self-Assembling Protein Arrays.

Ideally we would like to store many different self-assembling proteins together in a single library solution. It is therefore desirable that there be little to no exchange of the TerB sequence between fusion proteins. Given the dissociation rate of the Tus:Ter interaction, we can expect to be able to incubate clones together for several hours with negligible exchange of TerB sequences.

We can separately add either no, or an excess of, TerB-ZipCode2 to complexed GFP-Tus:TerB-ZipCode1 in solution, followed by binding of the mixture to cZipCode1 beads. If there is an exchange of Tus-bound TerB-ZipCode1 with solution-phase TerB-ZipCode2 then there should be a resulting loss of signal on the cZipCode1 bead. In a reverse experiment, we can add excess TerB-ZipCode1 to GFP-Tus:TerB-ZipCode2 and then bind the mixture to cZipCode1 beads. If there is an exchange, then we expect to see a gain of signal on cZipCode1 beads. As a series of non-limiting conditions, these tests can be repeated as a function of time (1, 2, 4, 6, 8, 10 and 24 hours), temperature (4° C., RT, 30° C. and 37° C.) and KCl concentration (0, 50, 100, 200 and 400 mM). (2) Solutions of Tus:TerB-ZipCode in either 20% glycerol or substitute cryogenic reagent can be flash-frozen in liquid Nitrogen and stored at least overnight at −80° C. The solutions can be thawed on ice. These solutions can again be used as above following different storage times and conditions to determine whether the protein remains active and the protein-DNA interaction retains integrity

Alternatively, to prevent randomization of the tagging sequences in a mixed population of tus-fusion moieties, we can keep the Tus:TerB reagents separate until ready to use. We can choose different cryogenic freezing reagents. We can use lyophilization techniques as a means for freeze-drying the proteins for long-term storage.

We do not have to store proteins as libraries for this disclosure to be successful. Most proteins can be frozen in 20% glycerol with or without various additives such as polyethylene glycol, DMSO, etc.

### Example 4

### Use of PLA to Measure Antigen Concentration Permits Measurement of Low Concentration of Antigens in Solution, Particularly in a Multiplex Format

Because no washing steps are required, and only sequential additions to the incubation of first the sample and then a ligation-PCR mixture, a homogenous PLA is suitable for automation in high-throughput applications. The high assay sensitivity will allow 1-μl sample aliquots to be monitored by proximity ligation, reducing sample consumption and enabling analysis of samples available only in very small amounts. Also, substantially less mAb is used per assay compared to standard ELISAs, and because all assays are expected to perform favorably at similar reagent concentrations, new assays do not require extensive optimization. The precision of proximity ligation is currently at the level of real-time PCR detection, but improved quantitative detection strategies for nucleic acids may offer a further increase in precision.

(1) Proximity probes can be composed of scFv1-Tus:Ter-ZipCode1 and scFv2-Tus:Ter-ZipCode2 complexes, wherein the two scFvs each recognize different epitopes on the same antigen. We can use scFv we have obtained against, as a non-limiting example, different pairs of epitopes of a target antigen, in a sandwich format using standard ELISA-type reactions with two different affinity tags. scFv that perform well in this format can be made into His6-Tus hybrids and the scFv and Tus activity validated as in Specific Examples 1 and 2. (2) PLA can be performed by incubating samples with proximity probes in 5-μl incubations for 1 h, before addition of a 45-μl mix containing components required for probe ligation and qPCR. The mix can contain 50 mM KCl, 20 mM Tris-HCl (pH 8.4), 2.5 mM MgCl2, 0.4 units of T4 DNA ligase (Amersham Pharmacia Biosciences), 400 nM bridge oligonucleotide, 80 μM ATP, 0.2 mM dNTPs, 0.5 μM primers, 200 nM probe for the 5′ nuclease assay, and 1.5 units of platinum Taq DNA polymerase (Invitrogen). After a 5-min ligation reaction at RT, the reactions can be treated with ExoIII for 1 hour, then heated at 65° C. for 15 minutes and treated with Nth and UDG (NEBiolabs) before being transferred to a qPCR instrument for temperature cycling: 95° C. for 2 min and then 95° C. for 15 sec and 60° C. for 60 sec, repeated 45 times (Applied Biosystems PRISM 7700 or 7000). (3) We can vary the concentration of the 4 components in the reaction; proximity partners, bridge oligonucleotide and antigen over a range of several orders of magnitude and repeat the assay as described. A synthetic P1-ZipCode1-ZipCode2-P2 oligonucleotide and TaqMan assay can be used as positive controls, where needed. (4) Optional steps in proximity ligation. We can optionally use a gap-filling step prior to ligation. SNP genotypers using MIPs use this step to increase specificity; we can use it in our system using the described controls. The ligated oligonucleotides may be more efficiently amplified if removed from the protein-nucleic acid complex. To release the ligated P1-ZipCode1-ZipCode2-P2 oligonucleotide from the complex we could use helicase. But as a helicase-alternative, we can synthesize Ter oligonucleotides with dUTP. Uracil-DNA glycosylase (UDG) and Endonuclease III (Nth, New England Biolabs) can be used for the efficient release of the P1-ZipCode1-ZipCode2-P2 oligonucleotide from the protein-nucleic acid complex to enable more reproducible qPCR.

Alternatives. (1) Assay Protocol. To determine whether the concentrations of proximity probes applied in the incubation should be adjusted on the basis of their affinities, we can calculate the expected signal over background over a range of dissociation constants. Gulberg found that with probes having Kd values between 0.1 and 10 nM, it is suitable to use a fixed low amount of both probes [Gulberg 2004]. However, enough probes should be used in the assay to generate a stable protein-independent background in a range where real-time PCR offers high precision. This is achieved with about 50-500 amplicons, corresponding to 5 to 25 pM of the proximity probes in a 5-μL incubation volume, ligated and amplified in 50 μL. (2) Reagent purity. Reagent purity is of importance for assay performance. Impurities derived from proximity probe generation, such as free mAbs and free oligonucleotides, can be removed by purification. High levels of free mAb are expected to reduce the signal by blocking probe binding, but lower levels are not harmful because the assay operates below target saturating conditions. By contrast, free oligonucleotides as well as proximity probes with inactive protein binders reduce assay performance by raising the background [Gulberg 2004]. The oligonucleotides used in proximity probes can be full length, and their sequences can be selected to avoid secondary structures that could prevent hybridization of the connector oligonucleotide and formation of inter-probe hybrids. (3) Length of proximity probes. We can optimize the length of the DNA fragment to be long enough to reach around the Tus-scFv-antigen-scFv-Tus complex to find the other end of the bridge oligo, while not being so long that it begins to approximate a free oligonucleotide floating in solution. We can vary the lengths of either one or both proximity probes from, as a non-limiting example, 10 to 60 nucleotides at 10 bases per trial. (4) Multiplexing detection. As more detection reactions are performed in parallel, the issue of mAb cross reactivity becomes an increasing problem limiting scalability. PLA coupled with MIP technology offers a solution to this problem if unique ligation junctions are used for each cognate proximity probe pair.

### REFERENCES

The disclosure of each reference cited is expressly incorporated herein.
Adler M. Immuno-PCR as a clinical laboratory tool. Adv Clin Chem. 2005; 39:239-92.
Barletta J. Applications of real-time immuno-polymerase chain reaction (rt-IPCR) for the rapid diagnoses of viral antigens and pathologic proteins. Mol Aspects Med. 2006; 27:224-53.
Bartel, P. L., Chien, C., Sternglanz, R. and Fields, S. (1993) In Hartley, D. A. (ed.), Cellular interaction in development: a practical approach. IRL Press, Oxford, pp. 153-179.
Bayer, E. A., H. Ben-Hur, Y. Hiller, and M. Wilchek. Post-secretory modifications of streptavidin. Biochem. J. 1989; 259:369-376.
Bendixen C, Gangloff S, Rothstein R. A yeast mating-selection scheme for detection of protein-protein interactions. Nucleic Acids Res. 1994; 22:1778-9.
Buckholz R G, Simmons C A, Stuart J M, Weiner M P. Automation of yeast two-hybrid screening. J Mol Microbiol Biotechnol. 1999; 1:135-40.
Dahl F, Gullberg M, Stenberg J, Landegren U, Nilsson M. Multiplex amplification enabled by selective circularization of large sets of genomic DNA fragments. Nucleic Acids Res. 2005; 33:e71.
Duggan L J, Hill T M, Wu S, Garrison K, Zhang X, Gottlieb P A. Using modified nucleotides to map the DNA determinants of the Tus:TerB complex, the protein-DNA interaction associated with termination of replication in Escherichia coli. J Biol Chem. 1995; 270:28049-54.
Duggin I G, Andersen P A, Smith M T, Wilce J A, King G F, Wake R G. Site-directed mutants of RTP of Bacillus subtilis and the mechanism of replication fork arrest. J Mol Biol. 1999; 286:1325-35.
Fan, Jian-Bing, M. S. Chee & K. L. Gunderson. Highly parallel genomic assays. Nature Reviews Genetics 2006; 7:632-644.
Figeys D. Adapting arrays and lab-on-a-chip technology for proteomics. Proteomics. 2002; 2:373-82.
Finley R L Jr, Brent R. Interaction mating reveals binary and ternary connections between Drosophila cell cycle regulators. Proc Natl Acad Sci USA. 1994; 91:12980-4.
Fredriksson S, Gullberg M, Jarvius J, Olsson C, Pietras K, Gustafsdottir S M, Ostman A, Landegren U. Protein detection using proximity-dependent DNA ligation assays. Nat Biotechnol. 2002; 20:473-7.
Fromont-Racine M, Rain J C, Legrain P. Toward a functional analysis of the yeast genome through exhaustive two-hybrid screens. Nat Genet. 1997; 16:277-82.
Gao X, Gulari E, Zhou X. In situ synthesis of oligonucleotide microarrays. Biopolymers. 2004; 73:579-96.
Giot, L., Bader, J. S., Brouwer, C., Chaudhuri, A., Kuang, B., Li, Y., Hao, Y. L., Ooi, C. E., Godwin, B., Vitols, E., Vijayadamodar, G., Pochart, P., Machineni, H., Welsh, M., Kong, Y., Zerhusen, B., Malcolm, R., Varrone, Z., Collis, A., Minto, M., Burgess, S., McDaniel, L., Stimpson, E., Spriggs, F., Williams, J., Neurath, K., Ioime, N., Agee, M., Voss, E., Furtak, K., Renzulli, R., Aanensen, N., Carrolla, S., Bickeihaupt, E., Lazovatsky, Y., DaSilva, A., Zhong, J., Stanyon, C. A., Finley, R. L., Jr, White, K. P., Braverman, M., Jarvie, T., Gold, S., Leach, M., Knight, J., Shimkets, R. A., McKenna, M. P., Chant, J., and Rothberg, J. M. A protein interaction map of Drosophila melanogaster. Science. 2003; 302:1727-1736.
Gullberg M, Fredriksson S, Taussig M, Jarvius J, Gustafsdottir S, Landegren U. A sense of closeness: protein detection by proximity ligation. Curr Opin Biotechnol. 2003; 14:82-6.
Gullberg M, Gustafsdottir S M, Schallmeiner E, Jarvius J, Bjarnegard M, Betsholtz C, Landegren U, Fredriksson S. Cytokine detection by antibody-based proximity ligation. Proc Natl Acad Sci USA. 2004; 101:8420-4.
Gullberg M, Gústafsdóttir S M, Schallmeiner E, Jarvius J, Bjarnegård M, Betsholtz C, Landegren U, Fredriksson S. Cytokine detection by antibody-based proximity ligation. Proc Natl Acad Sci USA. 2004; 101:8420-4.
Gustafsdottir S M, Schallmeiner E, Fredriksson S, Gullberg M, Soderberg O, Jarvius M, Jarvius J, Howell M, Landegren U. Proximity ligation assays for sensitive and specific protein analyses. Anal Biochem. 2005; 345:2-9.
Gustafsdottir S M, Schlingemann J, Rada-Iglesias A, Schallmeiner E, Kamali-Moghaddam M, Wadelius C, Landegren U. In vitro analysis of DNA-protein interactions by proximity ligation. Proc Natl Acad Sci USA. 2007; 104:3067-72.
Gustafsdottir, S. M., A. Nordengrahn, S. Fredriksson, P. Wallgren, E. Rivera, E. Schallmeiner, M. Merza, and U. Landegren. Detection of individual microbial pathogens by proximity ligation. Clin. Chem. 2006; 52:1152-1160.
Gustafsdottir, S. M., J. Schlingemann, A. Rada-Iglesias, E. Schallmeiner, M. Kamali-Moghaddam, C. Wadelius, and U. Landegren. In vitro analysis of DNA-protein interactions by proximity ligation. Proc. Natl. Acad. Sci. USA. 2007; 104:3067-3072.
Gutmann O, Kuehlewein R, Reinbold S, Niekrawietz R, Steinert C P, de Heij B, Lab Chip. 2005; 5:675-81.
Hardenbol P, Banér J, Jain M, Nilsson M, Namsaraev E A, Karlin-Neumann G A, Fakhrai-Rad H, Ronaghi M, Willis T D, Landegren U, Davis R W. Multiplexed genotyping with sequence-tagged molecular inversion probes. Nat Biotechnol. 2003; 21:673-8.
Hardenbol P, Yu F, Belmont J, Mackenzie J, Bruckner C, Brundage T, Boudreau A, Chow S, Eberle J, Erbilgin A, Falkowski M, Fitzgerald R, Ghose S, Iartchouk O, Jain M, Karlin-Neumann G, Lu X, Miao X, Moore B, Moorhead M, Namsaraev E, Pasternak S, Prakash E, Tran K, Wang Z, Jones H B, Davis R W, Willis T D, Gibbs R A. Highly multiplexed molecular inversion probe genotyping: over 10,000 targeted SNPs genotyped in a single tube assay. Genome Res. 2005; 15:269-75.
Jarvius M, Paulsson J, Weibrecht I, Leuchowius K J, Andersson A C, Wahlby C, Gullberg M, Botling J, Sjöblom T, Markova B, Ostman A, Landegren U, Söderberg O. In situ detection of phosphorylated PDGF receptor beta using a generalized proximity ligation method. Mol Cell Proteomics. 2007; 6:1500-9.
Kamada K, Horiuchi T, Ohsumi K, Shimamoto N & Morikawa K. Structure of a replication-terminator protein complexed with DNA. Nature, 1996; 383, 598-603.
Landegren U, Schallmeiner E, Nilsson M, Fredriksson S, Banér J, Gullberg M, Jarvius J, Gustafsdottir S, Dahl F, Söderberg O, Ericsson O, Stenberg J. Molecular tools for a molecular medicine: analyzing genes, transcripts and proteins using padlock and proximity probes. J Mol Recognit. 2004; 17:194-7.

Lee C V, Liang W C, Dennis M S, Eigenbrot C, Sidhu S S, Fuh G. High-affinity human antibodies from phage-displayed synthetic Fab libraries with a single framework scaffold. J Mol Biol. 2004; 340:1073-93.
Li, S., Armstrong, C. M., Bertin, N., Ge, H., Milstein, S., Boxem, M., Vidalain, P. O., Han, J. D., Chesneau, A., Hao, T., Goldberg, D. S., Li, N., Martinez, M., Rual, J. F., Lamesch, P., Xu, L., Tewari, M., Wong, S. L., Zhang, L. V., Berriz, G. F., Jacotot, L., Vaglio, P., Reboul, J., Hirozane-Kishikawa, T., Li, Q., Gabel, H. W., Elewa, A., Baumgartner, B., Rose, D. J., Yu, H., Bosak, S., Sequerra, R., Fraser, A., Mango, S. E., Saxton, W. M., Strome, S., Van Den Heuvel, S., Piano, F., Vandenhaute, J., Sardet, C., Gerstein, M., Doucette-Stamm, L., Gunsalus, K. C., Harper, J. W., Cusick, M. E., Roth, F. P., Hill, D. E., and Vidal, M. A map of the interactome network of the metazoan C. elegans. Science 2004; 303:540-543.
Lindskog M, Rockberg J, Uhlen M, Sterky F. (2005) Selection of protein epitopes for antibody production. Biotechniques. 2005; 38:723-7.
Link, D. R., S. L. Anna, D. A. Weitz, and H. A. Stone, 2004, Geometrically Mediated Breakup of Drops in Microfluidic Devices. Phys Rev Lett, 92: 054503.
Maarten A. Jongsma and Ralph H. G. M. Litjens. Self-assembling protein arrays on DNA chips by auto-labeling fusion proteins with a single DNA address. Proteomics 2006; 6:2650-2655.
MacBeath G, Schreiber S L. Printing proteins as microarrays for high-throughput function determination. Science. 2000; 289:1760-3.
Mizuta R, Mizuta M, Kitamura D. Atomic force microscopy analysis of rolling circle amplification of plasmid DNA. Arch Histol Cytol. 2003; 66:175-81.
Moorhead M, Hardenbol P, Siddiqui F, Falkowski M, Bruckner C, Ireland J, Jones H B, Jain M, Willis T D, Faham M. Optimal genotype determination in highly multiplexed SNP data. Eur J Hum Genet. 2006; 14:207-15. Erratum in: Eur J Hum Genet. 2006;14:976.
Mulcair M D, Schaeffer P M, Oakley A J, Cross H F, Neylon C, Hill T M, Dixon N E. A molecular mousetrap determines polarity of termination of DNA replication in E. coli. Cell. 2006; 125:1309-19.
Nedved M L, Gottlieb P A, Moe G R. CD and DNA binding studies of a proline repeat-containing segment of the replication arrest protein Tus. Nucleic Acids Res. 1994; 22:5024-30.
Neylon C, Brown S E, Kralicek A V, Miles C S, Love C A, Dixon N E. Interaction of the Escherichia coli replication terminator protein (Tus) with DNA: a model derived from DNA-binding studies of mutant proteins by surface plasmon resonance. Biochemistry. 2000; 39:11989-99.
Niemeyer C M, Sano T, Smith C L, Cantor C R. Oligonucleotide-directed self-assembly of proteins: semisynthetic DNA-streptavidin hybrid molecules as connectors for the generation of macroscopic arrays and the construction of supramolecular bioconjugates. Nucleic Acids Res. 1994; 22:5530-9.
Niemeyer, C. M., M. Adler, B. Pignataro, S. Lenhert, S. Gao, L. Chi, H. Fuchs, and D. Blohm. Self-assembly of DNA-streptavidin nanostructures and their use as reagents in immuno-PCR. Nucleic Acids Res. 1999; 27:4553-4561.
Schallmeiner, E., E. Oksanen, O. Ericsson, L. Spangberg, S. Eriksson, U. H. Stenman, K. Pettersson, and U. Landegren. Sensitive protein detection via triple-binder proximity ligation assays. Nat. Methods 2007; 4:135-137.
Seeman N C. DNA in a material world. Nature. 2003; 421:427-31.
Sheets, M. D., Amersdorfer, P., Finnern, R., Sargent, P., Lindqvist, E., Schier, R., Hemingsen, G., Wong, C., Gerhart, J. C. and Marks, J. D. Efficient construction of a large nonimmune phage antibody library: the production of high-affinity human single-chain antibodies to protein antigens. Proc. Natl. Acad. Sci. USA, 1998; 95:6157-6162.
Skokotas A, Hiasa H, Marians K J, O'Donnell L, Hill T M. Mutations in the Escherichia coli Tus protein define a domain positioned close to the DNA in the Tus:Ter complex. J Biol Chem. 1995; 270:30941-8.

Skokotas A, Wrobleski M, Hill T M. Isolation and characterization of mutants of Tus, the replication arrest protein of Escherichia coli. J Biol Chem. 1994; 269:20446-55.
Slentz-Kesler K, Moore J T, Lombard M, Zhang J, Hollingsworth R, Weiner M P. Identification of the human Mnk2 gene (MKNK2) through protein interaction with estrogen receptor beta. Genomics. 2000; 69:63-71.
Söderberg O, Gullberg M, Jarvius M, Ridderstråle K, Leuchowius K J, Jarvius J, Wester K, Hydbring P, Bahram F, Larsson L G, Landegren U. Direct observation of individual endogenous protein complexes in situ by proximity ligation. Nat Methods. 2006; 3:995-1000.
Söderberg O, Leuchowius K J, Kamali-Moghaddam M, Jarvius M, Gustafsdottir S, Schallmeiner E, Gullberg M, Jarvius J, Landegren U. Proximity ligation: a specific and versatile tool for the proteomic era. Genet Eng (N Y). 2007; 28:85-93.
Stoevesandt O., and M. J. Taussig. Affinity reagent resources for human proteome detection: Initiatives and perspectives. Proteomics 2007; 7:2738-2750
Taylor J D, Briley D, Nguyen Q, Long K, Iannone M A, Li M S, Ye F, Afshari A, Lai E, Wagner M, Chen J, Weiner M P. Flow cytometric platform for high-throughput single nucleotide polymorphism analysis. Biotechniques. 2001; 30:661-669.
Templin M F, Stoll D, Schrenk M, Traub P C, Vohringer C F, Joos T O. Protein microarray technology. Trends Biotechnol. 2002; 20:160-6.
Torigoe H, Maruyama A. Synergistic stabilization of nucleic acid assembly by oligo-N3′→P5′ phosphoramidate modification and additions of comb-type cationic copolymers. J Am Chem Soc. 2005; 127:1705-10.
Uetz, P., Giot, L., Cagney, G., Mansfield, T. A., Judson, R. S., Knight, J. R., Lockshon, D., Narayan, V., Srinivasan, M., Pochart, P., Qureshi-Emili, A., Li, Y., Godwin, B., Conover, D., Kalbfleisch, T., Vijayadamodar, G., Yang, M., Johnston, M., Fields, S., and Rothberg, J. M. A comprehensive analysis of protein-protein interactions in Saccharomyces cerevisiae. Nature 2000; 403:623-627.
Uhlén M, Björling E, Agaton C, Szigyarto C A, Amini B, Andersen E, Andersson A C, Angelidou P, Asplund A, Asplund C, Berglund L, Bergström K, Brumer H, Cerjan D, Ekström M, Elobeid A, Eriksson C, Fagerberg L, Falk R, Fall J, Forsberg M, Björklund M G, Gumbel K, Halimi A, Hallin I, Hamsten C, Hansson M, Hedhammar M, Hercules G, Kampf C. Larsson K, Lindskog M, Lodewyckx W, Lund J, Lundeberg J, Magnusson, Malm E, Nilsson P, Ödling J, Oksvold P, Olsson I, Öster E, Ottosson J, Paavilainen L, Persson A, Rimini R, Rockberg J, Runeson M, Sivertsson Å, Sköllermo A, Steen J, Stenvall M, Sterky F, Strömberg S, Sundberg M, Tegel H, Tourle S, Wahlund E, Waldén A, Wan J, Wernérus H, Westberg J, Wester K, Wrethagen U, Xu L, Hober S and Ponté F (2005) A human protein atlas for normal and cancer tissues based on antibody proteomics. Mol Cell Proteomics. 2005; 4:1920-32.
Uhlen M, Ponten F. Antibody-based Proteomics for Human Tissue Profiling. Mol Cell Proteomics. 2005; 4:384-393.
Wacker R, Schroder H, Niemeyer C M. Performance of antibody microarrays fabricated by either DNA-directed immobilization, direct spotting, or streptavidin-biotin attachment: a comparative study. Anal Biochem. 2004; 330:281-7.
Wang, Y., M. Moorhead, G. Karlin-Neumann, M. Falkowski, C. Chen, F. Siddiqui, R. W. Davis1, T. D. Willis and M. Faham Allele quantification using molecular inversion probes (MIP). Nucleic Acids Res. 2005; 33:183.

Yu A A, Savas T A, Taylor G S, Guiseppe-Elie A, Smith H I, Stellacci F. Supramolecular nanostamping: using DNA as movable type. Nano Lett. 2005; 5:1061-4.
Zengerle R, Daub M. Fast and reliable protein microarray production by a new drop-in-drop technique. Lab Chip. 2005; 5:675-81.
Zhu L, Koistinen H, Wu P, Narvanen A, Schallmeiner E, Fredriksson S, Landegren U, Stenman U H. A sensitive proximity ligation assay for active PSA. Biol Chem. 2006; 387:769-72.

1. A polymer comprising a plurality of monomers, each monomer comprising a non-covalent complex of:
a fusion protein and
a nucleic acid molecule,
wherein the fusion protein comprises a Tus protein according to SEQ ID NO: 5 and a polypeptide wherein the nucleic acid molecule comprises a Ter site according to SEQ ID NO: 7.

2. The polymer of claim 1 wherein the polymer is a homopolymer.

3. The polymer of claim 1 wherein the polymer is a heteropolymer.

4. The polymer of claim 1 wherein the fusion proteins in the plurality of monomers comprise identical polypeptides.

5. The polymer of claim 1 wherein the fusion proteins in the plurality of monomers comprise a plurality of polypeptides.

6.-16. (canceled)

17. The polymer of claim 1 wherein the polymer comprises a plurality of enzymes which function in an enzymatic pathway.

18. (canceled)

19. A method of assembling a polymer comprising a plurality of monomers, comprising the steps of:
ligating a plurality of monomers to each other using a DNA ligase enzyme, each monomer comprising a non-covalent complex of:
a fusion protein, and
a nucleic acid molecule,
wherein the fusion protein comprises a Tus protein according to SEQ ID NO: 5 and a polypeptide, and wherein the nucleic acid molecule comprises a Ter site according to SEQ ID NO: 7.

20.-24. (canceled)

25. The method of claim 19 further comprising attaching said polymer to a substratum.

26. The method of claim 19 further comprising introducing one or more single stranded nicks into the nucleic acid molecule of the ligated monomers.

27. A protein-DNA complex which comprises:
a fusion protein; and
a nucleic acid molecule;
wherein a first portion of the nucleic acid molecule is double stranded and a second portion of the nucleic acid molecule is single stranded;
wherein the first portion comprises a Ter sequence according to SEQ ID NO: 7 and the second portion comprises an addressing sequence of at least 6 nucleotides;
wherein the fusion protein comprises a Tus protein according to SEQ ID NO: 5 and a binding polypeptide.

28.-33. (canceled)

34. The composition of claim 27 wherein the binding polypeptide is selected from the group consisting of a ligand, a receptor, an enzyme, an enzyme substrate, an scFv fragment, and an enzyme inhibitor.

35. A method to measure a target molecule which can be bound by two distinct binding polypeptides, comprising:
mixing a first and a second binding polypeptides with a target molecule to form a mixture; wherein each binding polypeptide is part of a fusion protein with a Tus protein according to SEQ ID NO: 5, wherein the Tus protein is bound to a DNA molecule which comprises a double-stranded portion and a single-stranded portion, wherein the double-stranded portion comprises a Ter sequence according to SEQ ID NO: 7, wherein the single stranded portion comprises a tag sequence, wherein the tag sequence uniquely corresponds to the binding polypeptide;
adding a bridging oligonucleotide to said mixture under conditions in which complementary DNA single strands will form double strands; wherein the bridging oligonucleotide comprises a first and a second portion, wherein the first portion is complementary to the tag sequence of the first binding polypeptide and the second portion is complementary to the tag sequence of the second binding polypeptide, wherein the first and the second portion of the bridging oligonucleotide are separated by 0 to 6 nucleotides;
adding DNA ligase to said mixture, wherein said ligase joins 5′ and 3′ ends of nicked double-stranded DNA molecules to form an analyte DNA strand comprising a ligated junction between said first tag sequence and said second tag sequence;
amplifying the first tag sequence, the ligated junction, and the second tag sequence to form an amplified analyte DNA strand;
assaying to determine amount in the mixture of the amplified analyte DNA strand, wherein the amount of the amplified analyte DNA strand is related to the amount of the target molecule.

36.-40. (canceled)

41. A method for attaching an enzyme to a substratum, comprising:
attaching a nucleic acid molecule to a substratum by means of covalent or non-covalent coupling, wherein the nucleic acid molecule comprises a Ter sequence according to SEQ ID NO: 7;
forming a complex between the nucleic acid molecule and a fusion protein, wherein the fusion protein comprises a Tus protein according to SEQ ID NO: 5 and an enzyme.

42.-49. (canceled)

50. A method of forming an arrayed library of diverse protein-DNA complexes, comprising the step of:
mixing together one or more substrata comprising arrayed single stranded probes and a library of diverse protein-DNA complexes in which each complex comprises:
a fusion protein; and
a nucleic acid molecule;
wherein a first portion of the nucleic acid molecule is double stranded and a second portion of the nucleic acid molecule is single stranded;
wherein the first portion comprises a Ter sequence according to SEQ ID NO: 7 and the second portion comprises an addressing sequence;
wherein the fusion protein comprises a Tus protein according to SEQ ID NO: 5 and a binding polypeptide;
wherein each addressing sequence is complexed with a fusion protein comprising a unique binding polypeptide;
wherein the single stranded probes each comprise a sequence of at least 6 nucleotides which is complementary to an addressing sequence in the nucleic acid molecules; whereby upon mixing, the protein-DNA complexes bind to single stranded probes having complementary sequences.

51.-56. (canceled)
